(19) United States
(12) Patent Application Publication
(10) Pub. No.: US 2013/0274803 A1
(43) Pub. Date: Oct. 17, 2013

(54) Vertebral Fixation Apparatus for the Correction of Spinal Deformities

(71) Applicants: Mahamad Hamza Hilali Ideros Noordeen, London (GB); Hazem Bayoumi Elsebaie, Mohandeseen Giza (EG)

(72) Inventors: Mahamad Hamza Hilali Ideros Noordeen, London (GB); Hazem Bayoumi Elsebaie, Mohandeseen Giza (EG)

(21) Appl. No.: 13/818,940

(22) PCT Filed: Aug. 23, 2011
(86) PCT No.: PCT/GB11/01256

§ 371 Date: Jul. 8, 2013

(30) Foreign Application Priority Data
Aug. 24, 2010 (EG) 2010081427

(51) Int. Cl. A61B 17/70

(52) U.S. Cl. CPC A61B 17/7019
USPC 606/256

(57) ABSTRACT

Vertebral fixation apparatus (1) arranged to correct spinal deformities. The apparatus includes a spinal support assembly having at least first and second support segments (3), the first support segment (3) includes at least one articulated joint (15) that is arranged to enable first and second support segments (3) to move relative to one another and locking means (21;45) for selectively locking the articulated joint (15). The apparatus further includes connector means (9) for connecting the first and second support segments (3) together, and anchor means (5) for attaching the first support segment (3) to a first vertebra (7) and for attaching the second support segment (3) to a second vertebra (7).

This invention relates to vertebral fixation apparatus for correcting and fixation of spinal deformities, as well as fixation of other spinal pathologies, and the combination of the fixation apparatus and a setting tool.

Spinal deformities including scoliosis, kyphosis or kyphoscoliosis pose great challenges in their treatment. In progressive cases, usually the spine cannot be controlled by bracing or even casting, and the spine will grow in a manner accentuating the deformity with all its known consequences, leaving spinal correction, instrumentation and fusion the only available option for treatment.

The simplicity, efficacy and safety of spinal correction have been the main challenges of research since spinal instrumentation started. Many of the spinal fixation systems consist of screws that are threaded into the vertebral bodies either through the pedicles posteriorly or directly anteriorly into the bodies. In the treatment of most spinal pathologies we need to fix two or more vertebrae together. The screws which are inserted into the vertebrae are then connected together by solid bendable rods or plates.

In the last few decades, spinal fixation systems have focused on the screw design and the rod-screw junction which are variable in different spinal systems. Current spinal systems include either top loading or side loading screws either monoaxial or polyaxial, which are connected together by solid rods or plates. Developments of the design of rods have been mainly in the rod material including memory rods. All these systems have been used for segmental fixation and correction of the vertebral column, which includes rod rotation, translation of the spine to the rod and vertebral derotation in order to achieve a straight spine in the coronal plane with the least amount of vertebral rotation while maintaining a normal sagittal contour.

The disadvantages of the previously described systems are almost all due to the fact that, despite the achievements made in segmental fixation of the vertebrae, the known systems cannot provide true independent correction of each vertebra three dimensionally because the rods used to correct the spine are solid rods which are not easily bent. These solid rods are used to connect independently mobile spinal members and lack the necessary versatility.

There are many problems that can arise from the use of solid rods in the current vertebral fixation systems:
connecting a solid rod to a severely deformed spine can sometimes be extremely difficult especially in the maximum areas of deformity and rotation; this has led to the use of many additional complex implants and instruments including persuaders, extenders, polyaxial extended shoulders, polyaxial connectors and polyaxial bolts;
bringing the vertebral bodies and fixing them to a solid pre bent rod can generate significant forces at the screw bone interface, especially in the dorsal spine, in smaller children where the pedicle are smaller, and in poor bone quality leading to screw failures;
solid rods limit the ability to freely manipulate and mobilize each vertebra independently of the others as they cannot rotate and move except around the axis of a solid longitudinal rod with limited degree of freedom;
the solid rod prevents the free independent correction of the vertebrae in all 3 dimensional planes. In other words, any change, for example in the rod contour in the sagittal plane, can adversely affect and limit a change in a coronal plane, and vice versa;
the strength of the solid rods after bending can be overcome by the strength of the deforming forces of the spine during surgery, therefore it is sometimes difficult to maintain the desired contour of the rod. It is not uncommon to start insertion of the rod in a certain contour and finish the surgery with a different contour (for example loss of the kyphosis of the rod during surgery leading to hypokyphosis);
the difficulty in correcting or adjusting the spine once the rod is inserted and fixed to the spine: this can only be done through in-situ bending of the rods and has its own limitations and problems, including:
   it requires special rod material and sizes;
   it can cause notching of the rod affecting its resistance to failure;
   sometimes the space between the screws does not allow the introduction of the bending tools with ease, particularly in smaller children or in the mid and upper dorsal spine;
   the forces generated to bend a solid rod can overcome the strength of screw bone interface which can lead to implant failures;
   in-situ bending can correct only by translation without the ability to correct vertebral rotation directly;
   even translation correction can be difficult in certain planes; there is a known difficulty with creating kyphosis in the dorsal spine with in-situ bending which can cause the screws to pull out;
   the translation correction achieved in certain planes is limited and does not match the segmental nature and relative mobility of the spine which can be hindered by the strength and stiffness of solid rods; and
   once both the concave and convex rods are inserted and fixed, in-situ bending of solid rods becomes very limited leading to inability of doing the final adjustments.

Another alternative for spinal fixation is using rods to provide a chain of segments that are connected together end to end via an articulated joint to provide support to the spine. However the ability to set the segments in a desired shape when inserted is limited due to the manner in which the segments are connected together, manipulated and the manner in which they are locked into position. A further limitation of the known systems is that they are unable to adjust the positions and orientations of each of the individual segments within the chain by the small increments required in order to correct spinal deformities over time during surgical intervention. It will be appreciated by the skilled person that often many incremental adjustments are required over a period of time in order to move the spine from a deformed condition to its corrected (or nearly corrected condition). Therefore there is a need for a vertebra fixation system that is able to make the necessary independent incremental positional/orientational adjustments of each of the segments within a chain in order to manipulate individual vertebra in the required manner to address the spinal deformity being treated, and to easily fix the relative positions of the segments when the adjustments have been made. In some segmental systems the articulated joints are provided by assembling ball and socket components together at the time of use. While this provides some constructional flexibility it can lead to failure of the articulated joint since the loads applied thereto are relatively high. Furthermore, some known segmental systems have a very limited range of motion between segments due to the design of the articulated joints. This limits the user's ability to achieve the necessary movement required in order to correct spinal deformities.

One known system includes a plurality of bone screws, wherein each bone screw includes an aperture formed in its body. One of the screws is attached to each of the vertebra to be treated. The screws are oriented such that their apertures are substantially axially aligned and the segmental fixation device is attached to the spine by feeding it through the apertures. This is a so called top loading system. A drawback to this type of system is that it has a high profile from the spine, which is undesirable since it limits the paediatric population that it can be used with, especially when treating the kyphotic dorsal spine. Furthermore, because the segmented fixation system is threaded through the screws it significantly limits the possibility of adjusting each segment relative to an adjacent segment to achieve the desired spinal correction.

Accordingly the invention seeks to mitigate at least one of the aforementioned problems, or at least to provide an alternative arrangement to known systems.

According to one aspect of the invention there is provided vertebral fixation apparatus arranged to correct spinal deformities, said apparatus including a spinal support assembly having a plurality of support segments, said plurality of support segments including first and second support segments, the first support segment includes at least one articulated joint that is arranged to enable first and second support segments to move relative to one another, locking means for selectively locking the articulated joint, spacing means for spacing the first and second support segments apart, and anchor means, such as bone screws, for attaching the first support segment to a first vertebra and for attaching the second support segment to a second vertebra. The spacing means separates the first and second segments from each other and obviates the need for the segments to be connected directly together.

Advantageously at least the first and second support segments, and preferably all of the support segments, each include at least one formation that is arranged to receive an engagement part of a setting tool used to adjust the position and/or orientation of the first and second support segments. This enables each of the support segments so arranged to be adjusted by the tool, thereby enabling correction of the spinal deformity by manipulating the support segments relative to each other in any plane allowable by the articulated joint and/or anchor means, which may include rotation and/or angulation and/or translational movement between adjacent support segments.

Each formation is arranged such that the setting tool can be releasably attached to the support segments. The at least one formation preferably includes at least one of a male and female formation that is arranged to receive and engage the engagement part of the setting tool. Advantageously the formation can be complementary to the engagement part of the setting tool.

Advantageously the at least one formation can comprise a threaded hole formed in the segment, and preferably a plurality of threaded holes. The tool can include at least one threaded engagement member that is arranged to be screwed into the threaded hole to releasably connect with the support segment. When securely attached, the tool can be used to adjust the position and/or orientation of the support segment with respect to its associated vertebra and/or an adjacent support segment. For tools including first and second connector members, the segments can each include a plurality of threaded holes to enable each of the threaded connector members to releasably attach to the segment.

Advantageously the articulated joint can include a ball and socket type joint. This enables significantly greater movement between the first and second support segments. This arrangement is particularly useful at sites where the deformity is large.

Advantageously the second support segment can include an articulated joint. Advantageously the articulated joint is a ball and socket type joint.

Advantageously the spacing means can be connected to the ball in the articulated joint on the first support segment. Advantageously the spacing means can be connected to the ball in the articulated joint on the second support segment. This configuration of articulated joints provides more versatile relative movement between the first and second segments, and is a particularly useful arrangement in areas of severe deformity.

Advantageously the spacing means can include a threaded spacing member. The spacing member can include at least one of an external and an internal screw thread. Preferably the screw thread extends along substantially the full length of the spacing member. Advantageously the spacing member can be fixedly attached to at least one of the balls in the articulated joints of the first and second support segments. Advantageously the spacing member can be releasably attached to at least one of the balls in the articulated joints of the first and second support segments. For example, the spacing member can include an external screw thread and at least one of the balls can include a threaded bore arranged to receive one end of the spacing member. Alternatively at least one of the balls can include a second spacing member that protrudes therefrom that includes at least one of an internal and an external screw thread that is arranged to receive one end of the threaded spacing member. Each support segment can include a threaded bore in one end of the segment and the spacing member connects the first and second support segments together by insertion into the threaded bore.

Advantageously the spacing means can include a spacing mechanism arranged to adjust the separation between the first and second support segments. This enables a user to set the distance between the first and second support segments in a controlled manner and thereby providing compression and/or distraction between the associated vertebrae, independently of the support segment-anchor means interface. Advantageously the spacing mechanism can be arranged to continuously adjust the separation between the first and second support segments. This enables incremental adjustments to be made.

The spacing mechanism can include at least one threaded spacing member for controlling the separation between the first and second support plates, and at least one locking nut.

The spacing mechanism can include a first spacing member having an external screw thread, a second spacing member having an internal screw thread that is arranged to receive at least part of the first spacing member, and a turning member attached to one of the first and second spacing members and is arranged to drive relative movement between the first and second spacing member when the turning member is rotated.

The spacing mechanism can include a support frame.

Advantageously the spacing means can be uncoupled from at least one of the first and second support segments to enable the first and second support segments to be detached from one another.

Advantageously the locking means is arranged to provide a plurality of locking conditions to the articulated joint, said locking conditions including a fully unlocked condition, a partially locked condition, and a fully locked condition. The locking condition is typically selected by the user. In the fully unlocked condition the articulated joint can move freely. In the partially locked condition, the locking means provides a frictional load to the articulated joint but the joint can be moved if the user applies a sufficiently large load to the support segment to overcome the frictional load applied by the locking means. In the fully locked condition that joint cannot move. Unlike known systems, correction of the spine is achieved by independently controlling each of the vertebra when the corresponding bone anchor is fixed to its corresponding plate segment. Each support segment is manipulated in the desired way in all 3 dimensional planes with the least force possible. Unlike known solid rods this process can be easily repeated to adjust the correction needed many times by unlocking, partially locking and fully locking the or each articulated joint. This way it is possible to achieve the necessary segmental correction of the vertebral column by independently controlling each segment in all dimensions, for example when the articulate joint comprises a ball joint. This mobility enables the connection of the support segments to the bone anchors in almost any direction regardless of the position of the vertebra and hence the direction of the screw, with virtually no stress on the anchor-bone interface on insertion. Advantageously the support segments are inserted while the articulated joint(s) is loose so they will be easily mounted on the bone anchors. The segmental controlled correction of each segment is then undertaken and the or each articulation is then partially-locked to keep the acquired correction while allowing additional correction by further adjusting the required segments until the final desired correction is acquired and then the apparatus is fully locked.

Advantageously the apparatus can include a second locking means for selectively locking the articulated joint. The second locking means can include a nut mounted on the threaded spacing member, that is arranged to lock the orientation of the ball with respect to the socket when moved into engagement with the socket. Advantageously the second locking means can be arranged to provide a plurality of locking conditions to the articulated joint, said locking conditions including a fully unlocked condition, a partially locked condition, and a fully locked condition.

Advantageously the first and second support segments can each include an opening for receiving the anchor means. Advantageously each opening is elongate, which enables the position of the anchor means to be continuously adjustable with respect to the support segment along the opening. This enables contraction and/or distraction between the first and second support segments. Preferably each opening is arranged substantially parallel to a longitudinal axis of its support segment. Advantageously, the support segment can be arranged to pivot about the axis of the bone anchor. Typically only one bone anchor is used to connect the support segment to its associated vertebra. This enables the plate to rotate relative to the bone anchor, which is typically a screw.

Advantageously the first support segment can include a second articulated joint, preferably of the ball and socket type. Advantageously the first ball joint is located at a first end of the first support segment. Advantageously the second ball joint is located at a second end of the first support segment.

Advantageously the second support segment can include a second articulated joint, preferably of the ball and socket type. Advantageously the first ball joint is located at a first end of the second support segment. Advantageously the second ball joint is located at a second end of the second support segment.

Advantageously the apparatus can include at least one further support segment arranged according to at least one of the first and second support segments, and the spacing means includes at least one additional spacing member.

Advantageously the spacing means can include at least one additional spacing mechanism, to adjust the separation between adjacent support segments.

Advantageously at least one of the articulated joints can be prefabricated as an integral part of its support segment. This provides an arrangement wherein the socket provides a very strong housing for the ball, which prevents the joint from failing when loaded. Alternatively, the socket housing can be arranged to enable the ball to be removable therefrom, for example the housing can include a hole in its side to enable the ball to be inserted into the housing and removed therefrom. The housing can also include a slot to enable a spacer member that is pre-connected to the ball to be inserted into the housing and removed therefrom.

According to another aspect of the invention there is provided the combination of the vertebral fixation apparatus according to any configuration described herein and a tool for manipulating each of the plurality of support segments. This enables the position and / or orientation of each of the support segments to be adjusted by the tool, thereby enabling correction of the spinal deformity by manipulating the support segments relative to each other in any plane allowable by the articulated joint and/or anchor means, which can include rotation and/or translational movement between adjacent support segments.

Advantageously the tool is arranged to be releasably attached to each support segment individually.

The tool can include at least one segment engagement member that is arranged to engage the support segment being adjusted. The or each engagement member includes a threaded portion for connection with a complementary screw thread formed in the support segment.

This enables the engagement member to be screwed into the threaded hole in the segment to releasably connect the support segment. When securely attached, the tool can be used to adjust the position and/or orientation of the support segment with respect to its associated vertebra and/or an adjacent support segment. For tools including first and second engagement members, the segments can each include a plurality of threaded holes to enable each of the threaded connector members to releasably attach to the segment.

Advantageously the or each engagement member is substantially rigid.

Advantageously the tool includes a handle. The handle can be releasably or fixedly attached to the or each engagement member. The handle is arranged substantially perpendicularly to the or each engagement member.

Advantageously the first and second engagement members are arranged substantially parallel to one another. This provides a stable and strong tool when connected to the handle and segments.

According to another aspect of the invention there is provided vertebral fixation apparatus arranged to correct spinal deformities, said apparatus including a spinal support assembly having a plurality of support segments, at least one articulated joint that is arranged to enable first and second support segments to move relative to one another, and locking means for selectively locking the articulated joint. This aspect of the invention can include any of the features of the first and second aspects.

Embodiments of the invention will now be described by way of example only, with reference to the drawings, in which:

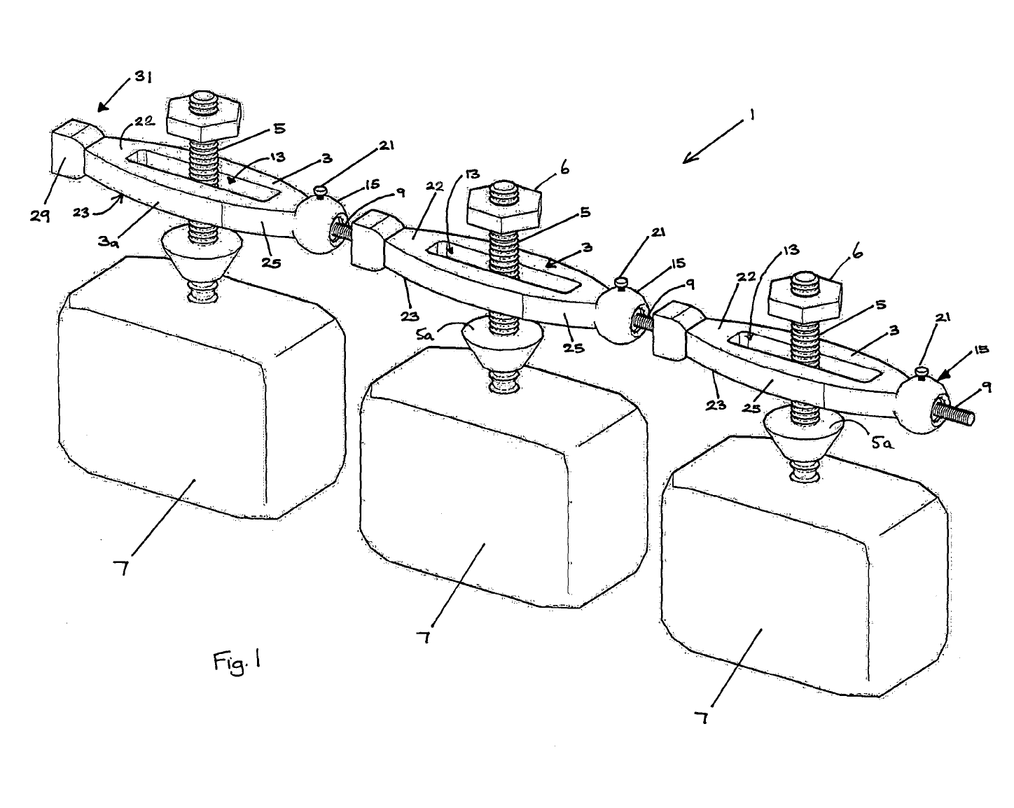

FIG. 1 is an isometric view of a first embodiment of the invention attached to vertebrae;

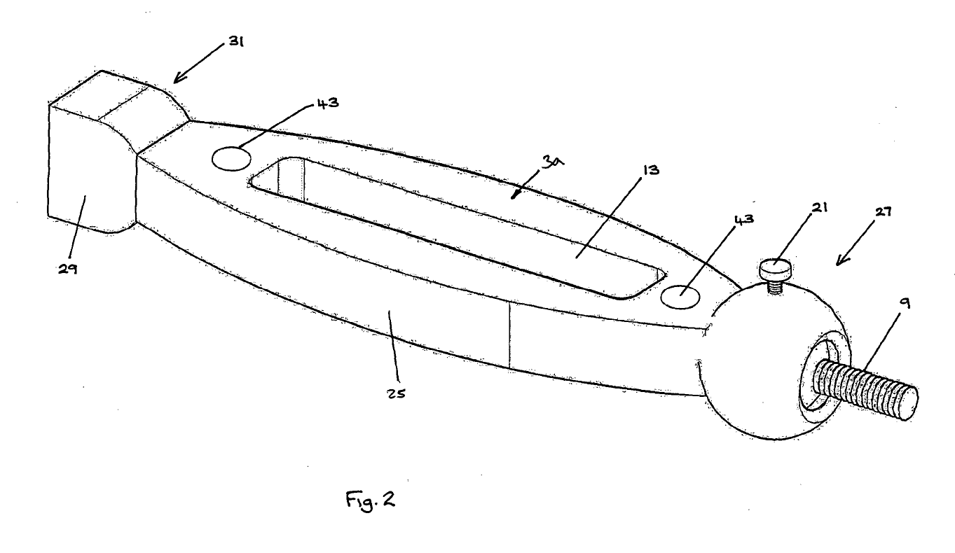

FIG. 2 is an isometric view of a single support plate used in the first embodiment including a ball joint and a primary locking mechanism for locking the orientation of the joint;

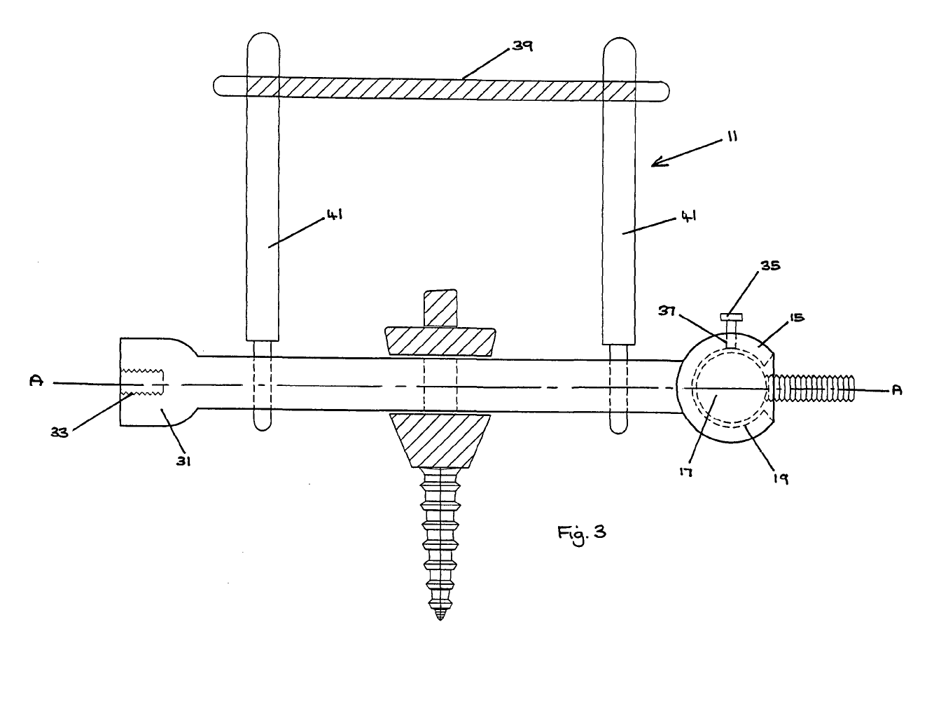

FIG. 3 is a side view of the support plate of FIG. 2 together with a tool that is used to adjust the orientation of the support plate;

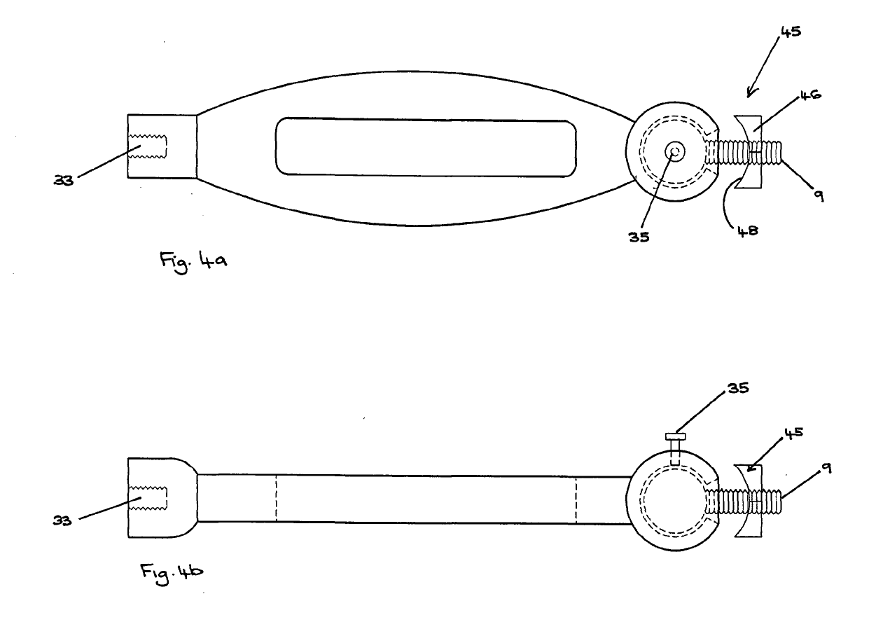

FIG. 4a is plan view of the support plate of FIG. 2 including an optional secondary locking mechanism;

FIG. 4b is a side view of the support plate of including the optional secondary locking mechanism;

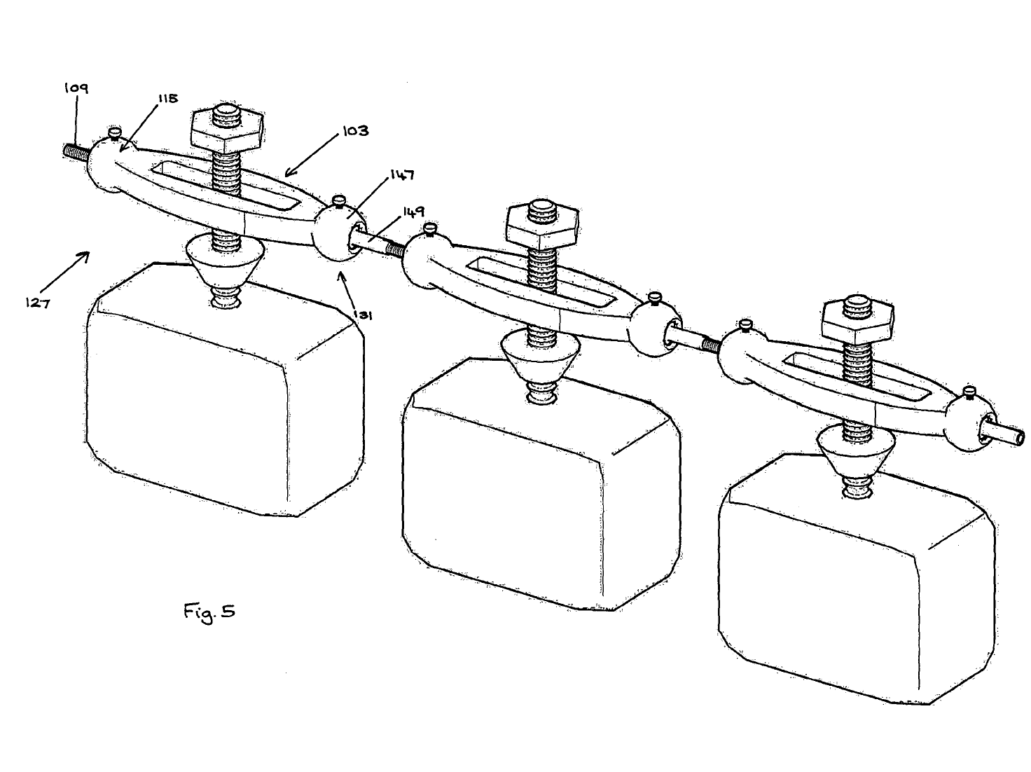

FIG. 5 is an isometric view of a second embodiment of the invention attached to vertebrae;

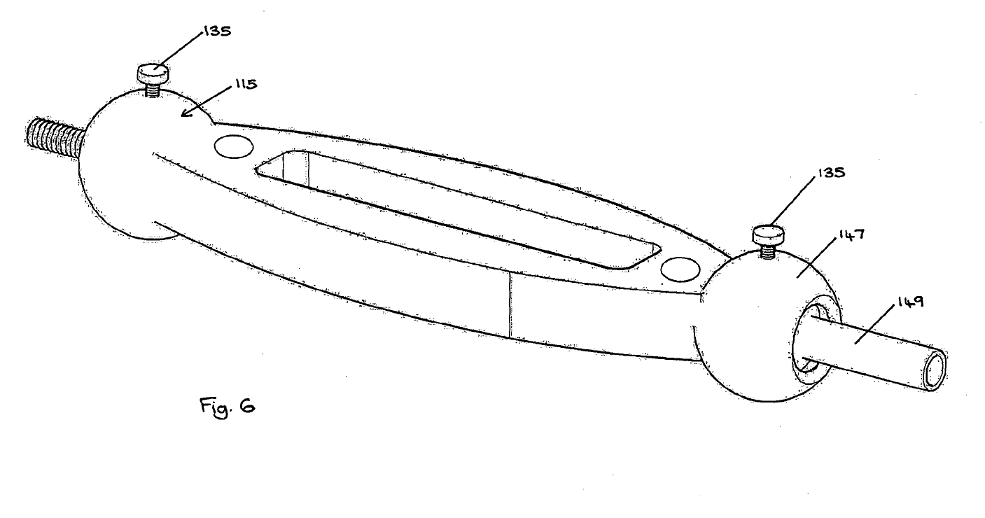

FIG. 6 is an isometric view of a single support plate used in the second embodiment, which includes first and second ball joints, each having a primary locking mechanism for locking the orientation of the joint;

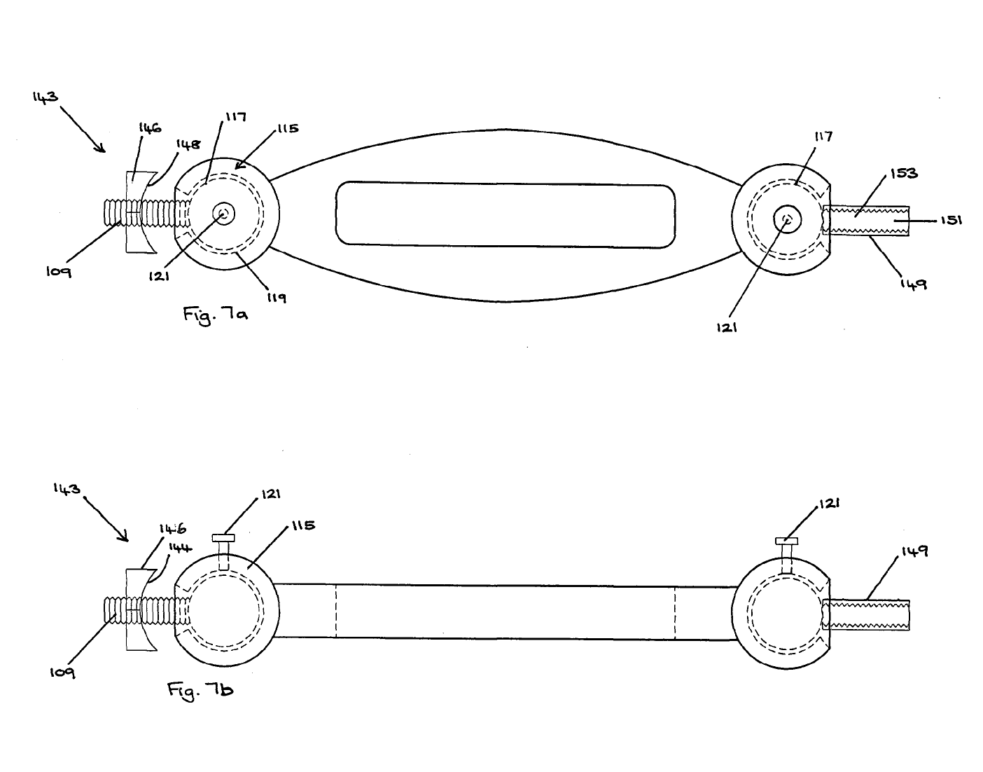

FIG. 7a is a plan view of the support plate of FIG. 2 including an optional secondary locking mechanism for one of the ball joints;

FIG. 7b is a side view of the support plate of FIG. 2 including the optional secondary locking mechanism for one of the ball joints;

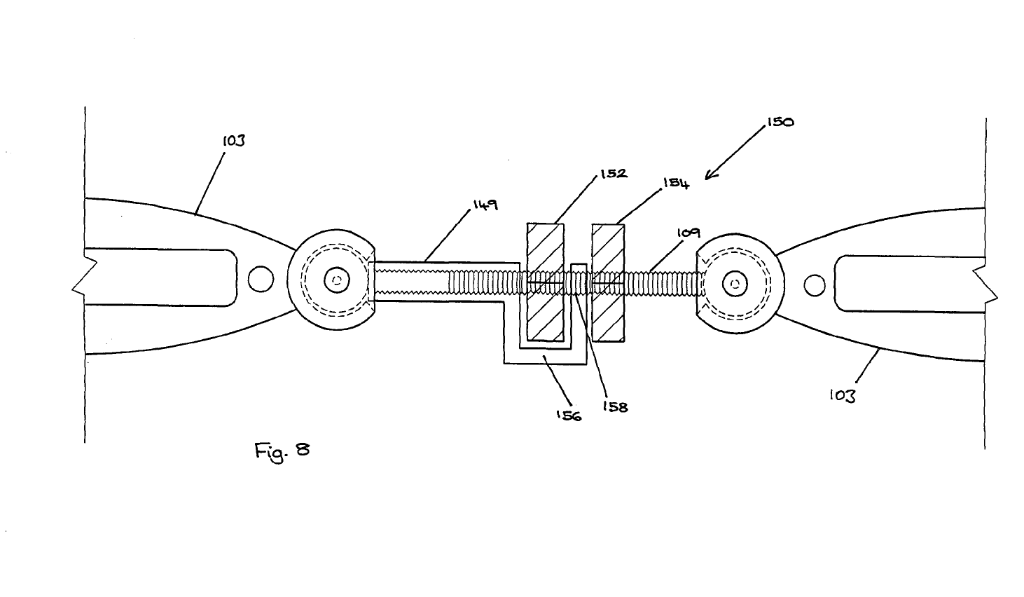

FIG. 8 shows a plan view of an optional mechanism for adjusting the separation between adjacent support plates;

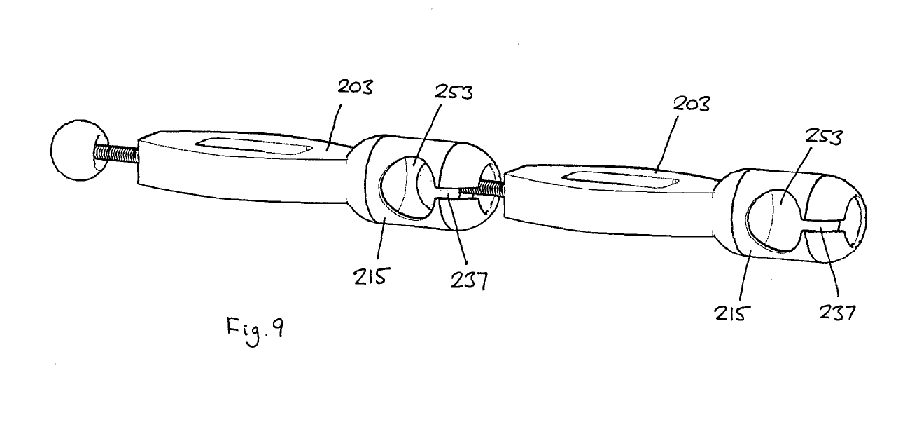

FIG. 9 shows an isometric view of a third embodiment of the invention; and

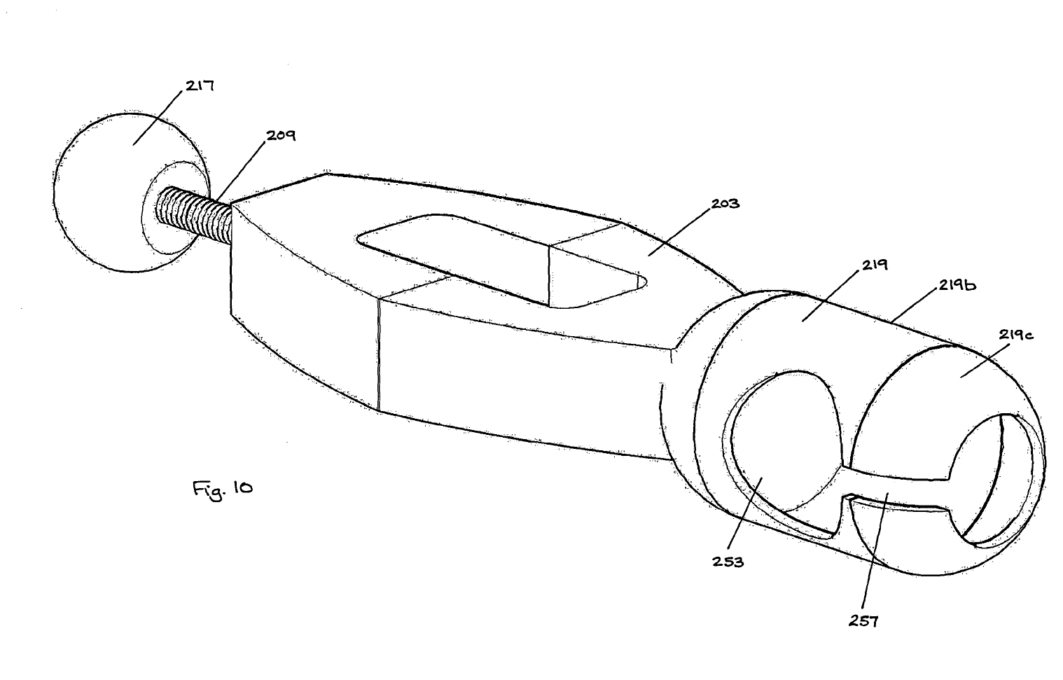

FIG. 10 is an isometric view of a support plate used in the third embodiment of the invention.

FIGS. 1 to 4b show a spinal support apparatus 1 according to a first embodiment of the invention. The apparatus 1 includes a plurality of vertebra support plates 3 (hereinafter referred to as support plates 3), a plurality of bone screws 5 for attaching individual support plates 3 to individual vertebra 7 (shown diagrammatically in FIG. 1), and a plurality of spacing elements 9 for connecting adjacent support plates 3 together.

Each support plate 3 is similar and includes a slot 13, a ball joint 15 comprising a ball 17 and socket 19, and a locking mechanism 21 for selectively locking the ball joint 15 by locking the orientation of the ball 17 relative to the socket 19. The apparatus 1 is used for correcting spinal deformity by providing a chain of support plates 3 with inter segmental articulations between the support plates allowing motion in all 3 dimensional planes (same as a ball and socket or any similar articulation) and at the same time allowing selective graded locking of these articulations.

Each support plate 3 is elongate and includes a central body 3a having a longitudinal axis A-A (see FIG. 3). The central body 3a includes first and second planar surfaces 22,23 and convex side walls 25 that provide a substantially elongate elliptical shape to the central body 3a when viewed in plan. The slot 13 is formed through the central body 3a, and provides openings in the first and second planar surfaces 22,23. The slot 13 is arranged along the longitudinal axis A-A and extends along a substantial part of the length of the body 3a. The slot 13 is substantially rectangular and has rounded ends, when viewed in plan.

The slot 13 is arranged to receive a bone screw 5. The bone screw 5 is typically a posted pedicle screw.

A distal end of the bone screw 5 is inserted into a vertebra 7 and the support plate 3 is mounted on the bone screw 5 such that it abuts a shoulder portion 5a. The position of the support plate 3 relative to the vertebra 7, and hence compression and distraction between adjacent vertebrae, can be adjusted by moving the support plate 3 relative to the bone screw 5, which moves within the slot 13. The position of the support plate 3 is fixed relative to the screw 5 by tightening a nut 6, which is located towards a proximal end of the bone screw 5. When fixed, the screw 5-support plate 3-vertebral body 7 acts as a single unit and any motion of the support plate 3 in any direction is directly transmitted to the vertebral body 7 therefore allowing a truly segmental control and correction of the spine by independently controlling each support plate 3 and hence vertebra 7. Adjustment of the position and/or orientation is achieved by unlocking the nut 6 and allowing the bone screw 5 to move within the slot 13.

The arrangement of the support plate 3 provides a relatively thin profile when compared with prior art devices, which enables the device to have a wider application, and is particularly useful in children and at the kyphotic dorsal spine. Also the nut 6 impinges on the first planar surface 22 when in its locked condition and therefore is located within the overall thickness of the support plate 3. If necessary a recess (not shown) can be formed in the first planar surface 22 to further reduce the profile if required. Furthermore the arrangement of the slot 13 in body 3a enables each plate to be mounted on the posted screws in a versatile and easy way even with the presence of severe deformity. This contrasts with the known vertically mounted screw rod systems where the rod has to be mounted within the groove in the screw head, which may need some forceful manoeuvres until the rod is seated completely. The posted screws 5 and the slots 13 enable the support plates 3 to be mounted on the screws 5 in a much simpler manner.

The support plate 3 segments are fabricated in different sizes to fit with different inter vertebral distances (i.e. the upper dorsal has a different size from lower lumbar). The length of slot 13 allows distraction and compression between corresponding screws 5 and hence the vertebrae as needed, for example in levelling of the concave and convex pedicles at the distal or proximal ends of the construct. The slots 13 can also have different lengths to account for the variable inter vertebral distances, for example to accommodate for different patients ages, sizes, different levels of the spine from cervical to lumbar with variable and inter-pedicular distances.

The ball joint 15 is located at a first end 27 of the support plate. A block 29 is located at a second end 31 of the support plate. A threaded bore 33 is formed in the block 29. The bore 33 is arranged so that it is substantially aligned with the longitudinal axis A-A of the support plate. The bore 33 is arranged to receive a first end of the threaded spacing element 9 of an adjacent support plate to connect the adjacent support plates together.

The threaded spacing element 9 includes an external screw thread that preferably extends along substantially the full length of the spacing element 9. The spacing element 9 is fixed to the ball 17 towards a second end protrudes out of the socket 19 by a predetermined distance. As well as connecting adjacent support plates 3 together, the spacing element determines the distance between the first end 27 of one support plate 3 and the second end 31 of an adjacent plate 3.

The ball joint 15 is prefabricated such that the ball 17 is located within the socket 19. This enables the opening in the socket to be smaller than the size of the ball 17 thereby preventing the ball from dissociating itself from the socket 19, when loaded. Therefore the risk of the ball joint 15 failing in use is greatly reduced.

The ball joint 15 enables adjacent plates to be moved relative to one another. The ball joint allows the orientations to be adjusted in 3 dimensions by a limited amount, which is defined by the shape and size of the socket 19 opening. The ball joint 15 enables the segmented chain to bend in all planes as well as for each segment to rotate about is longitudinal axis. Typically the threaded spacing element 9 can move within a conical space, wherein the angle a defining the slope of the conical space is typically in the range 60-120°.

The ball joint 15 provides a wide range of motion between adjacent support plates 3 in all directions as well as rotation of the support plate 3 around its longitudinal axis A-A.

The locking mechanism 21 includes a screw element 35 that is arranged to impinge on the ball 17 via a threaded bore 37 formed in the socket casing 19. The screw element 35 can selectively lock the orientation of the ball 17 relative to the socket 19 by engaging the ball 17. The ball 17 can be fully unlocked, partially locked and fully locked by the screw element 35. When the screw 35 does not engage the ball 17, the ball is free to move with respect to the socket 19 and therefore is fully unlocked. When the screw element 35 is rotated into engagement with the ball 17 and applies some load to the ball 17 but not sufficient to fully lock the orientation of the ball 17, the ball 17 is said to be in a partially locked state. The partially locked state maintains the relationship between the adjacent support plates 3 while still allowing mobility of the articulation when a certain force is applied to change the position, thereby allowing further adjustment. In this condition the user can approximately set the positions and/or orientations of the adjacent support plates 3 and then can undertake fine adjustments by applying a sufficient load to the support plate 3 in order to overcome the resistive force of the locking screw 35. When the screw 35 is tightened further such that the ball 17 cannot move relative to the socket 19, the ball 17 is fully locked in position and it is necessary for the user to release the screw 35 in order for further adjustments to be made. The partial lock is a useful feature since it provides a first stage of locking which enables the user to lock the support plate 3 in a position that is approximately correct. The user is then free to adjust the positions of other support plates 3, each time subsequently partially locking the ball joints 15. When all of the plates have been adjusted, the chain of plates will be in approximately the correct position and there will be some stiffness within the chain. Final adjustments can then be made in the partially locked state to refine the positions of the support plates 3, where this is possible. If necessary, the user can fully unlock one or more of the ball joints 15 in order to finalise the positions.

Thus the locking mechanism 21 provides variable locking forces in three degrees of motion of the ball joint.

The tool 11 is used to adjust the positions and/or orientations of each of the support plates 3. The tool 11 includes a handle 39 and two arms 41 (see FIG. 3). The arms 41 can be releasably connected to the handle 39 at their proximal ends and include external screw threads at their distal ends so that they can be releasably attached to each of the support plates 3 via threaded holes 43 formed in the body 3a (the holes 43 are shown in FIG. 2 but have been omitted from other drawings for clarity purposes). In use, the arms 41 are screwed into the holes 43 and the handle 39 is connected to the proximal ends thereof. When fully attached, the user can use the tool to finely adjust the position and/or orientation of support plates by manipulating the tool 11. Since the arms 41 of the tool are relatively long they provide the user with the ability to make fine adjustments. When a support plate 3 has been adjusted, the tool 11 is detached from that support plate and is then used to adjust the other support plates 3 in the chain, as required.

Additionally, or alternatively, the tool 11 can be adapted to engage and manipulate the support plate via the slot 13.

FIGS. 4a and 4b show a second locking system 45 that can be used in addition to, or as an alternative to the first locking mechanism 21. The second locking mechanism 45 includes a nut 46 mounted on the threaded connector 9. The nut 46 includes a concave inner surface 48 that is arranged to engage the socket 19 when tightened in order to restrain the movement of the ball 17. The nut 46 is able to engage the socket casing 19 since the external screw thread on the spacing element 9 extends to, or terminates close to, the ball 17. The nut 46 can be arranged to partially and fully lock the orientation of the ball 17 with respect to the socket 19, according to the locking load applied. The nut 46 can also be fully disengaged to enable the ball 17 to rotate freely with respect to the socket 19.

FIG. 5 shows a second embodiment of the invention. The second embodiment is similar to the first embodiment except that the block 29 is replaced with a second ball joint 147 at the second end 131 of the support plate 103. The second ball joint 147 is similar to the first ball joint 115, as described in relation to the first embodiment, except that it has a second spacing element 149 attached to the ball 117, wherein the second spacing element 149 includes an axial bore 151 having an internal screw thread 153. The second spacing element 149 is arranged to receive the first spacing element 109 of an adjacent support plate 103 to enable the support plates 3 to be connected together and to be spaced apart by a distance defined by the arrangement of the spacing elements 109,147.

Alternatively to the second spacing element 149, the ball of the second ball joint 147 can include a threaded bore in its body.

The second embodiment provides greater mobility between the support plates 103. This arrangement is particularly useful for the treatment of severely deformed areas of the vertebral column like the apex of the deformity, where it is desirable to have an increased degree of mobility with a short support plate 3 between two adjacent pedicles.

FIGS. 7a and 7b show a second locking mechanism 143 that can be used in addition to, or as an alternative to the first locking mechanism 121 used on the first ball joint 115. The second locking mechanism 143 includes a nut 146 mounted on the threaded connector 109. The nut 146 includes a concave inner surface 148 that is arranged to engage the socket 119 when tightened in order to restrain the movement of the ball 117. When the nut 46 can be arranged to partially and fully lock the orientation of the ball 117 with respect to the socket 119, according to the locking load applied. The nut 146 can also be fully disengaged to enable the ball 117 to rotate freely with respect to the socket 119.

FIG. 8 shows a mechanism 150 that is arranged to adjust the separation between adjacent support plates 103, and therefore provides an additional means of providing compression and/or distraction between adjacent support plates 103. The mechanism 150 is arranged to provide continuous adjustment of the separation between the adjacent support plates 103. The mechanism 150 includes an adjusting nut 152, a locking nut 154, the second spacing element 149 in modified form, which includes a u-shaped frame member 156 having a threaded hole 158 formed therein. The spacing element 109 of an adjacent support plate 103 is inserted into the threaded bore of the second spacing element 149 via the threaded hole 158. The distance between the adjacent support plates 103 is adjusted by rotating the adjustment nut 152, which causes the first threaded spacing element 109 to move into/out of the threaded bore thereby adjusting the separation between the adjacent support plates 103. When the final position has been reached, the position is locked by tightening the locking nut 154.

FIGS. 9 and 10 show an alternative support plate 203 arrangement. Instead of providing a prefabricated ball joint 115, the ball joint 215 can include a hole 253 an a slot 257 formed in the side of the socket 219 casing. The hole 253 and slot 257 enable the ball 217 and spacing element 209 of an adjacent support plate 203 to be inserted into the socket 219 and removed therefrom.

The invention is relatively easy to fabricate and is easy to use. It can be readily used in fixing different types of spinal pathologies and deformities through:
Posterior pedicle screw / hook fixation.
Anterior vertebral body screw fixation.

The uses of the invention include correction and fixation of spinal deformities including scoliosis and kyphosis, spinal fixation for different pathologies including degenerative spinal conditions, tumours, fractures, etc, or as a type of dynamic fixation which provides some types of motion between vertebral bodies.

One of the most important ways of using the system is in correcting spinal deformities. The bone screws are inserted in the vertebrae using conventional tools. The chain of support plates 3,103,203 are connected to the bone screws 5 in a condition wherein the ball joints are loose. The choice of the type, number, and size of the support plates 3,103,203, and the size of the slots 13 are tailored to each patient (a template support plate can be used for this purpose). Each support plate 3,103,203 is mounted on its respective bone screw 5 such that the screw 5 is located within the slot 13. The support plate 3,103,203 is fixed to its bone screw 5 by the nut 6. The position/orientation of each support plate 3,103,203 is then manipulated by the tool 11. This movement transmits a load to the corresponding vertebral body via the bone screw 5. Each support plate 3,103,203 is manipulated in relation to an adjacent support plate 3,103,203 by rotating and translating the support plates relative to each other until the required relation between them is achieved. This can be achieved starting either with the concave or convex sides of the spine, proximally, distally or at the apex, as best determined by the user. The joint(s) between the two segments are then partially locked to partially fix the relationship while further adjustments and corrections are carried out without losing the correction once the force is relieved.

The adjustment of the support plates 3,103,203 is repeated proximally and distally until the deformed vertebral column is corrected and stays in the position achieved by the serial manipulations. The process controls and manipulates each vertebra in 3 dimensional planes and then locks it in the required position. If distraction or compression between screws 5 is needed, it achieved by undoing the nuts 6 fixing the screws 5 to the support plates 3,103,203 thereby allowing distraction and/or compression as required by moving the screws 5 within their respective slots 13 of the corresponding support plates. When the continuous adjustment mechanism 150 is used, direct compression and/or distraction between adjacent support plates 3,103,203 can be achieved by adjusting the separation between the adjacent support plates 3,103,203 by controlling the adjustment mechanism 150. When the final desired correction is achieved the ball joints are fully locked in position by final tightening the locking mechanism(s).

The invention offers many advantages in correcting spinal deformities compared to the conventional systems:
the support plates 3,103,203 are easier to manipulate than a solid rod;
the support plates 3,103,203 each include a specific formation for connecting with a manipulation tool, for ease of manipulation, mechanical advantage and better control;
the process can be repeated many times with ease through locking, partially locking and unlocking the ball joints;
the shape of the support plates 3,103,203 themselves are arranged to provide an additional correction factor by fabricating its shape in a curved way which can add kyphosis or lordosis as needed; and
it can be used in dynamic fixation allowing motion between vertebral bodies.

It will be appreciated by the skilled person that modifications can be made to the above embodiments that fall within the scope of the invention, for example the mechanism 150 for adjusting the separation between the support plates 103 can be adapted for use in the other embodiments disclosed.

An alternative adjustment mechanism can be used to the adjustment mechanism 150 for adjusting the separation between adjacent support plates, and hence contraction and/or distraction of the associated vertebrae, for example a spacing element having an adjustable length such as in a telescopic arrangement can be used to control the separation between the adjacent support plates.

A mixture of the support plates of the above embodiments can be included in a single apparatus and can be adapted to be connected together as required.

1. Vertebral fixation apparatus arranged to correct spinal deformities, said apparatus including a spinal support assembly having a plurality of support segments, said plurality of support segments including first and second support segments, the first support segment includes at least one articulated joint that is arranged to enable first and second support segments to move relative to one another, locking means for selectively locking the articulated joint, spacing means for spacing the first and second support segments apart, and anchor means for attaching the first support segment to a first vertebra and for attaching the second support segment to a second vertebra.

2. Apparatus according to claim 1, wherein at least the first and second support segments each include at least one formation that is arranged to receive an engagement part of a setting tool used to adjust the position and/or orientation of the first and second support segments.

3. Apparatus according to claim 2, wherein formation is arranged such that the setting tool can be releasably attached to support segments.

4. Apparatus according to claim 3, wherein the at least one formation includes a threaded hole formed in the segment, and preferably a plurality of threaded holes.

5. Apparatus according to claim 1, wherein the articulated joint includes a ball and socket type joint.

6. Apparatus according to claim 1, wherein the second support segment includes an articulated joint.

7. Apparatus according to claim 6, wherein the, articulated joint is a ball and socket type joint.

8. Apparatus according to claim 5, wherein and the spacing means is connected to the ball in the articulated joint on the first support segment.

9. Apparatus according to claim 8, wherein the second support segment includes an articulated joint that is a ball and socket type joint, and the spacing means is connected to the ball in the articulated joint on the second support segment.

10. Apparatus according to claim 8, wherein the spacing means includes a threaded spacing member.

11. Apparatus according to claim 1, wherein the spacing means includes a spacing mechanism arranged to adjust the separation between the first and second support segments.

12. Apparatus according to claim 11, wherein the spacing mechanism is arranged to continuously adjust the separation between the first and second support segments.

13. Apparatus according to claim 11, wherein the spacing mechanism includes at least one threaded spacing member for controlling the separation between the first and second support plates, and at least one locking nut.

14. Apparatus according to claim 13, including a first spacing member having an external screw thread, a second spacing member having an internal screw thread that is arranged to receive at least part of the first spacing member, and a turning member attached to one of the first and second spacing members that is arranged to drive relative movement between the first and second spacing member when the turning member is rotated.

15. Apparatus according to any one of claim 11, wherein the spacing mechanism includes a support frame.

16. Apparatus according to claim 1, wherein the spacing means can be uncoupled from at least one of the first and second support segments to enable the first and second support segments to be detached from one another.

17. Apparatus according to claim 1, wherein the locking means is arranged to provide a plurality of locking conditions to the articulated joint, said locking conditions including a fully unlocked condition, a partially locked condition, and a fully locked condition.

18. Apparatus according to claim 1, including a second locking means for selectively locking the articulated joint.

19. Apparatus according to claim 18, wherein the spacing means includes a threaded spacing member, and the second locking means includes a nut mounted on the threaded spacing member, that is arranged to lock the orientation of the ball with respect to the socket when moved into engagement with the socket.

20. Apparatus according to claim 1, wherein the first and second support segments each include an opening for receiving the anchor means.

21. Apparatus according to claim 1, wherein the second support segment includes a second articulated joint, preferably of the ball and socket type.

22. Apparatus according to claim 1, wherein the first support segment includes a second articulated joint, preferably of the ball and socket type.

23. Apparatus according to claim 1, including at least one further support element arranged according to at least one of the first and second support elements, and the spacing means includes at least one additional spacing member.

24. Apparatus according to claim 23, wherein the spacing means includes at least one additional a spacing mechanism.

25. Apparatus according to claim 1, wherein at least one of the articulated joints is prefabricated as an integral part of the support segment.

26. The combination of the vertebral fixation apparatus of claim 1 and a tool for manipulating each of the plurality of support segments.

27. The combination according to claim 26, wherein the tool is arranged to be releasably attached to each of the support segments.

28. The combination according to claim 27, wherein the tool includes at least one segment engagement member that is arranged to engage the support segment being adjusted.

29. The combination according to claim 28, wherein the or each engagement member includes a threaded portion for connection with a complementary screw thread formed in the support segment.

30. The combination according to claim 28, including first and second engagement members, wherein the first and second engagement members are arranged substantially parallel to one another.

31. The combination according to claim 28, wherein the or each engagement member is substantially rigid.

32. The combination according to claim 26, wherein the tool includes a handle.
